(19) United States
(12) Patent Application Publication
(10) Pub. No.: US 2020/0299628 A1
(43) Pub. Date: Sep. 24, 2020

(54) THERMALLY RESPONSIVE MICROGEL PARTICLES FOR CELL CULTURE APPLICATIONS

(71) Applicant: Battelle Memorial Institute, Columbus, OH (US)

(72) Inventors: Anthony D. Duong, Columbus, OH (US); Jacob L. Lilly, Marble Cliff, OH (US)

(21) Appl. No.: 16/821,022

(22) Filed: Mar. 17, 2020

Related U.S. Application Data
(60) Provisional application No. 62/820,159, filed on Mar. 18, 2019.

(51) Int. Cl. C12M 1/12; C12N 5/074; C12M 3/06; C12M 1/00; C12M 1/34

(52) U.S. Cl. CPC C12M 25/14; C12N 5/0696; C12M 41/12; C12M 23/28; C12M 23/16

(57) ABSTRACT

Thermo-responsive microgel particles can be used to provide a thermally triggered liquid-like solid (LLS) support scaffold for immobilizing and growing three-dimensional cell cultures. Various applications and devices using such microgel particles, and methods of using such microgel particles, are also described.

This application claims priority to U.S. Provisional Patent Application Ser. No. 62/820,159, filed Mar. 18, 2019.

### BACKGROUND

The present disclosure relates to thermally responsive microgel particles for use in in vivo cell culture; methods for cultivating cell cultures or structures using the microgel particles, and devices employing the microgel particles. The present disclosure describes particular applications for thermo-responsive microgel particles that can provide a tunable viscosity for anchoring and yielding cell cultures. However, it is to be appreciated that the present disclosure is also amenable to other like applications.

Cell culture dishes (e.g. Petri dishes) are widely employed for in vitro propagation of cultured cells. The cells adhere to the substrate, which is in the form of a flat surface, which provides the cells with mechanical support and access to nutrients. However, cell culture dishes cannot simulate the mechanical and chemical gradients—which control cell shape and structure—found in vivo. Instead, cells grow in two-dimensional (2D) monolayers, which result in homogenous growth and proliferation.

A 3D cell culture is a recent alternative to the cell culture dish. A naturally derived extracellular matrix (ECM), such as collagen or Matrigel®, can form a 3D polymer network or scaffold for cells to grow. These networks can generate macroscopic structures that generally mimic physiological structures, but they cannot generate the cellular interactions that are critical to functional biological systems. Synthetic materials also exist, but they may cause non-specific immunogenicity if implanted in a host. Furthermore, the mechanical properties of both natural and engineered matrices prevent the precise placement or structuring of cells in the 3D space; impede cell movement through a scaffold microenvironment; and rely on a cellular self-assembly that limits the macroscopic structure that can be generated.

A 3D growth medium that allows for precise placement of cells in a 3D space while further enabling the cells to grow into a predefined structure is desirable.

### BRIEF DESCRIPTION

The disclosure is directed to thermo-responsive packed microgel particles, which provides a thermally triggered liquid-like solid (LLS) support for 3D cell cultures and immobilization. Such particles allow for precise placement of cells in a 3D space while further enabling the cells to grow into a predefined structure. The viscosity of the microgel particles can be tuned, which can be useful for separating the cellular structure from the media. The microgel particles also exhibit improved flow and deformation under applied force.

Disclosed in various embodiments herein are microgel particles comprising a thermally responsive hydrogel matrix. The hydrogel matrix comprises a thermo-responsive polymer that expands at a threshold temperature, which can be, in some examples, about 37° C. The hydrogel matrix may behaves as a Bingham plastic, and sometimes can yield under a stress of about 10 Pascals (Pa). In some embodiments, the hydrogel matrix is selected to have a yield stress greater than about 10 Pa. In other embodiments, the hydrogel matrix is selected to have a yield stress below about 10 Pa. In still others, the hydrogel matrix is selected to have a yield stress from about 5 Pa to about 15 Pa. The microgel particles may further include at least one of a signaling molecule, growth factor, protein, exosome, cell lysate, or antigen (collectively “inducing molecules”) for inducing a cellular process in cells that come into contact with the inducing molecules on the microgel particles.

Also disclosed in various embodiments herein are cell culture systems. The cell culture systems include a bioreactor chamber that contains a liquid-like solid (LLS) growth media. A scaffold is formed from a plurality of microgel particles seeded into the LLS growth media. The LLS growth media itself may be formed from the same or different microgel particles. The microgel particles forming the scaffold comprise a thermally responsive hydrogel matrix. Mammalian cells are dispersed throughout the scaffold. The mammalian cells grow to form the cellular structure.

Also disclosed herein are methods for generating a cell structure having a predefined orientation, profile or shape. The methods include the step of seeding a plurality of microgel particles into a liquid-like solid (LLS) growth media to form a scaffold within the LLS growth media. The seeded microgel particles comprise a thermally responsive hydrogel matrix and mammalian cells. The mammalian cells grow to form the cellular structure.

Also disclosed herein in various embodiments are cell culture systems that include a microfluidic device containing microgel particles in at least one chamber of the microfluidic device. The microgel particles are comprised of a thermally responsive hydrogel matrix. Cells, mixed with the microgel particles, are trapped or released by controlling the temperature of the microgel particles in the chamber. The cells can be expanded in the hydrogel matrix to form a cell culture.

Also further disclosed are various methods for generating an in vitro cell culture. In a microfluidic device containing a chamber of thermally responsive microgel particles, cells are delivered to the chamber when a temperature of the microgel particles is below a threshold temperature, which in one example can be about 37° C. The microgel particles comprise a hydrogel matrix that expands at the threshold temperature. The methods include the step of raising a temperature of the chamber to above the threshold temperature. This causes the matrix to expand, trapping cells in place, for example between expanded microgel particles. While the cells are trapped, they expand (i.e. reproduce) within the hydrogel matrix/scaffold created by the microgel particles to form a cell culture. After cell expansion, the temperature of the microgel particles is lowered to below the threshold temperature to allow for removal of the cell culture (i.e. expanded cells) from the chamber.

These and other non-limiting aspects of the present disclosure are described in more detail below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a schematic showing the thermoresponsive microgel particles of the present embodiment being employed as a 3D culture media at different temperatures. On the left, the microgel particles are used as a scaffold for expanding cells at an elevated temperature. On the right, at a lower temperature, the microgel particles shrink and are easily separated from the cells.

FIG. 2 is a graph showing the hydrodynamic diameter of the microgel particles versus temperature. The y-axis is in nanometers, and runs from 200 nm to 400 nm at intervals of 40 nm. The x-axis is in degrees Celsius, and runs from 5° C. to 45° C. at intervals of 5° C. The curve is sigmoidal.

FIG. 3 is a schematic showing another application where the microgel particles are used as a scaffold in a LLS 3D culture media for forming functional nerve structures.

FIG. 4 is a microfluidic device capable of performing multiple steps using the microgel particles for transfection and cell expansion.

FIG. 5 is another embodiment of a microfluidic device that employs the microgel particles in a chamber with local heating and cooling loops for selective release of cells over time.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

The present disclosure may refer to temperatures for certain method steps or properties of polymers. It is noted that these generally refer to the temperature at which the heat source (e.g. furnace) is set, and do not necessarily refer to the temperature which must be attained by the material being exposed to the heat.

Some terms used herein are relative terms. For example, the terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the fluids flow through an upstream component prior to flowing through a downstream component. It should be noted that in a loop, a first component can be described as being both upstream of and downstream of a second component.

The present disclosure relates to various applications that are enabled through the use of microgel particles that are thermally responsive. In this regard, microgel particles can provide a robust medium for 3D fabrication of delicate structures. For example, soft granular microgels can be formed, for example from cross-linked poly-(acrylic acid) copolymers, to have a mesh size that allows for nearly unimpeded diffusion of nutrients. Packed granular microgel particles can form a liquid-like solid (LLS) material that freely diffuses to occupy a given volume, but also exhibit elastic properties that recover quickly. Gentle yielding and rapid solidification of a LLS 3D culture medium allows for unrestricted placement and structuring of cells and cell-assemblies deep within the medium.

The mechanical properties of LLS growth media enable cell division to occur with negligible physical resistance. In other words, the LLS provides cradling support, but does not physically impede cell division. Where cells require a stiffer microenvironment, an ECM can be mixed into the LLS medium. However, cells exhibit different migration behaviors in the polymer networks and LLS. In LLS, granular gel is displaced in the direction of travel and spontaneously flows to fill the space behind the cells. In ECM, migration is enabled through permanent tunnels through enzymatic degradation, making ECMs undesirable anchors for cells in the LLS.

The microgel particles of the present disclosure can be used as a LLS medium. Specifically, the disclosure is directed to liquid-like, thermally responsive microgel particles that provide a solid support growth medium for 3D cell growth. The microgel particles can be used as a liquid-like solid (LLS), and can be adopted to grow or expand numerous cell types including mammalian cells or bacteria cells, among others, etc. These microgel particles allow for nearly unimpeded cell movement through a scaffold microenvironment, a property not afforded by other technologies. The microgel particles can be displaced with forces as low as one piconewton (1 pN), which his on the order of actin polymerization and cytoskeletal rearrangement. The void spaces between particles allows for rapid nutrient transport that cannot be achieved with conventional crosslinked hydrogels. The size, composition, and degree of crosslinking in the microgel particles can be selected to meet the conditions necessary to support the growth of the specific cell type.

In embodiments, the microgel particles have a particle size on the order of a mammalian cell, or from about 10 μm to about 100 μm. However, the microgel particles do not need to be limited by these particle sizes.

Generally, the microgel particles comprise a thermally responsive hydrogel matrix. Hydrogels are hydrophilic, three dimensional cross-linked polymer systems capable of imbibing large amounts of water or biological fluids between their polymeric chains to form aqueous semi-solid/solid gel networks. The disclosure contemplates a hydrogel made from loose cross-linkages, which create an interparticle space(s) for nutrients to diffuse and cells to move.

The present disclosure further allows for specific inducing molecules to be linked (covalently bonded) to the hydrogel matrix of the microgel particles. Alternatively, the liquid-like nature of the 3D matrix allows for the inducing molecules (whether organic or inorganic) to be suspended in the liquid or interpenetrate the liquid-like solid. In particular embodiments, such inducing molecules can be one of a signaling molecule, growth factor, protein, exosome, cell lysate, or antigen. It is contemplated that such inducing molecules can contact the cells as the cells move throughout the hydrogel matrix/scaffold, activating specifically targeted cellular functions. The inducing molecules can induce at least one cellular process, such as growth; differentiation; activation; taxis; transport; mitosis; apoptosis; or a combination of the above. The inducing molecules can be selected to stimulate cellular growth at a specific rate or spatial orientation. Alternatively, the hydrogel matrix can be selected with specific cross-linkages and interparticle spacing to control or accelerate cellular growth at a desired rate.

In some embodiments, the cells can be tagged with IHC staining, fluorescent or luminescent protein labels. In this manner, fluorescence and confocal imaging systems can be used to measure the growth of the cells in the hydrogel matrix scaffold formed by the microgel particles in the LLS 3D growth medium and to characterize a morphology of the cells (e.g. size, aspect ratio, and/or cell alignment). If the cells are neural cells, the electrophysiological activity of nerve structures can be further analyzed in vitro and compared to the expected physiology of a typical nerve structure. Biomolecular and mechanical cues can be optimized to further support cell growth. For example, for cells that are being expanded for future implantation in a human patient, a panel of inflammatory cytokines can be measured via ELISA to evaluate immune response. Lactate Dehydrogenase (LDH) assays can be used to determine in vivo cytotoxicity.

In particular embodiments, the hydrogel matrix comprises a thermo-responsive polymer that expands at a threshold temperature, which can also be referred to herein as a “critical solution (CS)” temperature. The thermo-responsive polymer can have an upper critical solution temperature (UCST) or a lower critical solution temperature (LCST) between about 25° C. and about 40° C., and more preferably about 37° C., although there is no limitation made herein to the exact temperature. The hydrogel matrix/thermo-responsive polymer responds to temperature changes relative to the threshold temperature.

Some monomers that may be present in the thermo-responsive polymer include N-isopropylacrylamide; 2-(dimethylamino)ethyl methacrylate; vinylcaprolactame; hydroxypropylcellulose; N-acryloylglycinamide; N-vinylimidazole; 1-vinyl-2-(hydroxylmethyl)imidazole; and vinyl methyl ether. The thermo-responsive polymer may contain other monomers as well. The hydrogel matrix may also comprise other polymers which are not thermo-responsive, but which are cross-linked with the thermo-responsive polymer.

In some embodiments, the cell culture system may be cooled when sufficient cell expansion and/or manipulation has been achieved. Upon lowering a temperature of the cell culture system to below the threshold temperature (i.e., cooling), the LLS contracts and allows the media to become a flowable liquid. This can permit rapid, efficient harvesting of the expanded cell culture.

In preferred embodiments, the hydrogel matrix behaves as a Bingham plastic. A Bingham plastic is a viscoplastic material that behaves as a rigid body at low stresses, but does not flow until a yield stress is reached, at which point it flows as a viscous fluid at high stress. Preferably, the microgel particles have a dual phase behavior, allowing them to have a high volume, nutrient and gas transport of a liquid, but a 3D structure and mechanical cues of a solid.

Desirably, the microgel particles can be displaced with a protrusion force of about 1 piconewton (pN), which is on the order of actin polymerization and cytoskeletal rearrangement of the cells in vivo. Also desirably, the hydrogel matrix yields under a stress of about 10 Pascals (Pa), including from about 5 Pa to about 15 Pa. Thus, the hydrogel matrix has a liquid-like state that allows cells to move throughout the matrix and nutrients to diffuse to the cells. As the cells move through the matrix, they encounter microparticles or molecules that are dispersed within the matrix.

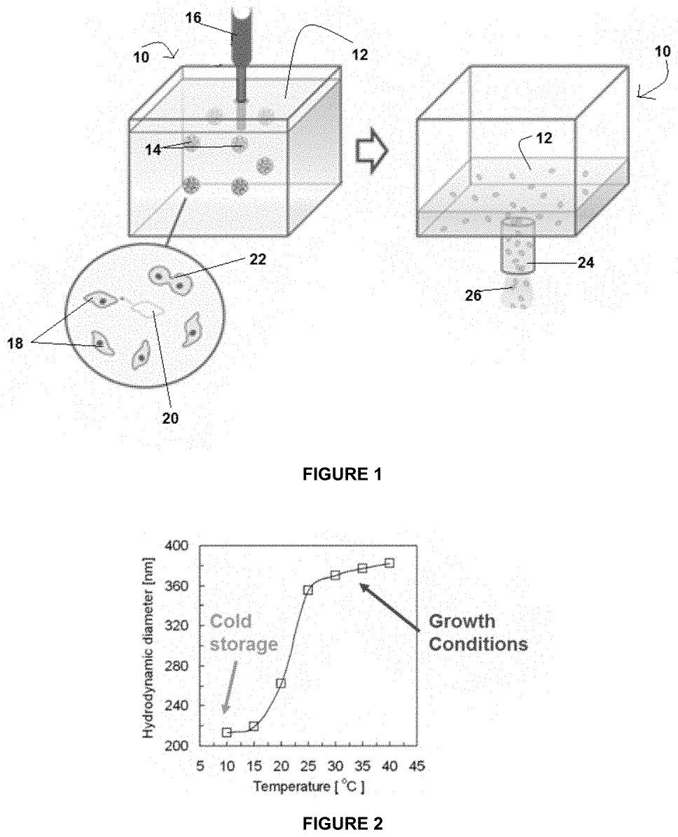

FIG. 1 is a schematic showing one application of the thermo-responsive microgel particles as a 3D culture media at different temperatures. On the left-hand side is a bioreactor chamber 10 containing a LLS 3D growth medium 12. The bioreactor 10 is capable of achieving scalable, reproducible manufacture of cell structures, and can be employed to simultaneously produce multiple structures in parallel. Several microgel particles 14 are seeded into the LLS growth media 12 to form a scaffold(s) at a desired location(s) with the chamber 10. The microgel particles 14 comprise a thermally responsive hydrogel matrix, and are different from the 3D LLS growth media 12. The scaffold in the illustrated embodiment is shown as a sphere, but there is no limit made herein to the shape of the scaffold.

The hydrogel matrix/microgel particles 14 can be placed into the LLS by a 3D printing process. A concentrated pellet is loaded into a syringe 16, which is used to pipette or inject the microgel particles 14 into the LLS growth media 12. 3D printing gives the ability to arrange cells into complex, spatially organized structures. The self-healing nature of the microgel particles allows for the automated printing of virtually any geometry or defined spatial orientation, and in a way that can be easily scaled outward.

In the illustrated embodiment, the scaffold is spherical. In certain embodiments, processing parameters, such as printing speed/flow rate, print head geometry (core/sheath diameter) are controlled to produce scaffold structures of various sizes, shapes, and morphologies. Conditions including gas and nutrient exchange, seeding densities, and other parameters can be further optimized to maximize the production efficiency and rate for generation of the cell assembly or structure.

The exploded view of FIG. 1 shows the microparticles 14 with cells 18. After the cells are dispersed into the hydrogel matrix, the temperature of the microgel particles is selectively raised to above the threshold temperature. This causes the hydrogel matrix of the microgel particles to expand and form a scaffold. The cells can move through the scaffold, promoting cellular propagation. As the cells multiple or expand to grow new cells, they grow from the scaffold in a predetermined orientation. As the cells expand, the cell culture generally conforms to the shape of the scaffold. In contemplated embodiments, the scaffold can be defined to mimic the structure of a host organoid tissue that the cellular structure or assembly will replace.

Continuing with the right-hand side of FIG. 1, when the bioreactor achieves a sufficient cell expansion and/or manipulation, the temperature of the bioreactor can be lowered to below the threshold temperature. By lowering the temperature, the hydrogel matrix/scaffold of the microgel particles 14 contract or liquefy, releasing water. It is believed that this also causes the cells to be separated from the matrix/scaffold. The liquid containing the microgel particles and the cells can be drained (shown via port 24) from the chamber 10. The thermo-responsive quality of the microgel particles enable the expanded cells 26 to be separated from the microgel particles with reduced risk of damage to the cells.

FIG. 2 is a graph showing the hydrodynamic diameter of one example of the microgel particles relative to temperature. As shown in the graph, the hydrodynamic diameter increases disproportionally with temperature, forming a sigmoidal curve. As temperature increases, the microgel particles expand to occupy more space. In the example graph, the hydrodynamic diameter is about 220 nm at temperatures below about 15° C. The hydrodynamic diameter increases sharply between about 15° C. and 25° C. At cell growth conditions, the hydrodynamic diameter is larger. At lower temperatures, i.e. cold storage, the hydrodynamic diameter is smaller. In this graph, the hydrodynamic diameter increases from about 220 nm at 10° C. to about 380 nm at 35° C.

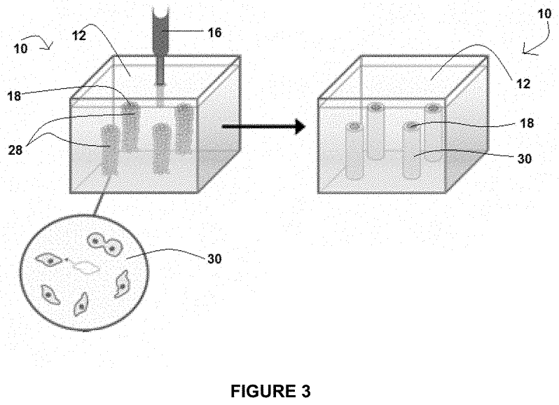

Turning to FIG. 3, a schematic illustrates another application, in which the microgel particles are used to form nerve structures. The bioreactor chamber 10 contains a liquid-like solid (LLS) growth media 12. A plurality of microgel particles 14 are seeded into the LLS growth media 12 using a syringe 16, to form a scaffold 28 within the LLS growth media.

In some embodiments, the syringe can be used to inject a printing ink that contains both the microgel particles and cells 18. In others embodiment, the microgel particles are first deposited in the LLS media 12 to form the scaffold 28, and the cells 18 are subsequently dispersed throughout the scaffold 28, which is illustrated as a core-sheath structure 28 in FIG. 3. The cells 18 propagate, as shown in exploded view 30, to form the cellular structure having a shape and profile that is similar to, and influenced by, the seeded scaffold pattern.

The growth factor pre-seed strategy can be used to promote growth patterns, cell polarities, and microstructure partitioning that is unattainable by traditional growth matrices.

The present disclosure does not limit use of the microgel particles to bioreactors. Additional embodiments are contemplated which incorporate the microgel particles into smaller devices.

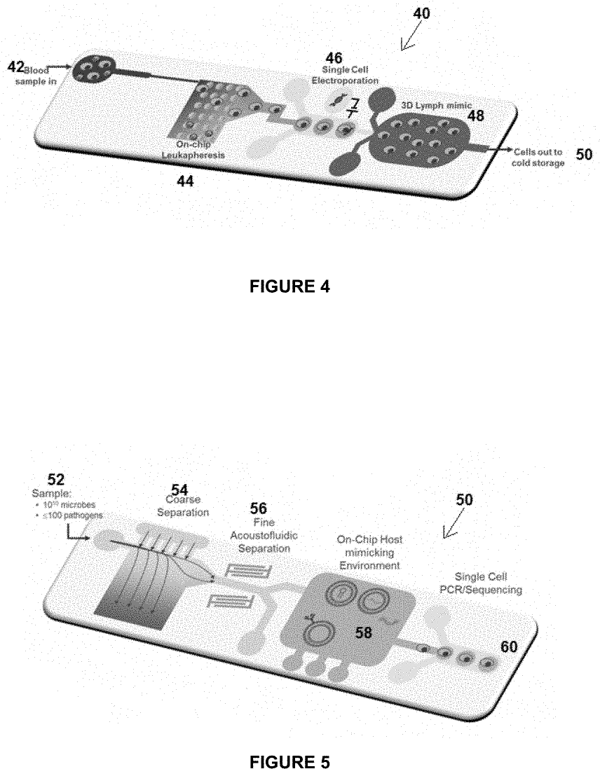

Turning to FIG. 4, another embodiment is contemplated which uses the microgel particles in a continuous flow mesofluidic/microfluidic device 40 for rapidly separating, transfecting, and expanding a cell. In this capacity, “mesofluidic” describes a fluidic flow device involving channels with dimensions of about 100 micrometers (μm) or larger.

The illustrated device 40 has a generally planar profile and can be formed from a polymeric material, although there is no limit herein made to the shape, material or manufacture of the device. The device has a length, width, and height/thickness/depth. In the illustrated embodiment, the fluidic device 40 includes at least one chamber containing the microgel particles. In other embodiments, the fluidic device 40 may include an array of multiplexed chambers, each containing the microgel particles. It is contemplated that cells will be delivered to the chamber(s). The temperature may then be increased to physiological temperature, at which point the microgel particles expand and form a scaffold for the cells. The microgel particles may be functionalized to interrogate, activate, or expand the cells (i.e. through the use of inducing molecules as previously described). By localizing the temperature (i.e., heating and cooling) within each chamber, specific chambers within the device 40 may be heated or cooled to release cells of interest in a programmable fashion.

In greater detail, the device 40 shown in FIG. 4 can be used to combine and automate several labor and time intensive steps in a therapy that requires, at one step, cell expansion. For illustrative purposes, the fluidic device is shown for use with CAR-T therapy and T-cells, but there is no limit made herein to the type of cell, therapy, or sub-processes being used with or performed by the device.

The device 40 includes at least one input channel 42 for accepting an input sample into the device. In one embodiment, a blood sample can be delivered to the device 40 using the input channel 42. The blood flows to a microfilter(s) or ratchet section 44 that fractionates the whole blood into individual components (macrophages, erythrocytes, etc.). Put another way, the microfilter section 44 performs on-chip leukapheresis. The microfilter exploits the size and shape differences between cell types to sort and separate the cell types, e.g., the T-cells, leukocytes, macrophages, erythrocytes, etc. In one embodiment, asymmetrical flow field-flow fractionation (AF4), which employs microchannels and pulsatile flow, can be used to separate the different cells based on membrane rigidity and fidelity, etc. Finer cell separation can be performed using acoustofluidics.

Next, the fractionated T-cells/lymphocytes can be encapsulated in a single water-in-oil emulsion droplet and transfected with a nucleotide encoding for the CAR of interest (e.g., anti-CD19 in the case of cancer treatment). The transfection can be performed via nanochannel electroporation (NEP) at an electroporation section 46. Others input into the electroporation section can include the water-in-oil emulsion and the nucleotide. The transfected T-cells would then be transported to the mesofluidic chamber 48 (via delivery channels in the device 40) containing the thermo-responsive LLS microgel particles. The chamber 48 is at a temperature that allows the transfected cells to mix with the microgel particles. The temperature is raised to the physiological temperature to allow for solidification and cell expansion of the hydrogel matrix/scaffold of the particles, at which point the chamber acts as an on-chip host mimicking environment. When the cell culture has matured, the temperature is reduced to shrink the microgel particles and to allow the cells to be delivered from the device 40 via an output channel 50.

In other contemplated embodiments, the microgel particles can include specific antigens that activate the T-cells. The liquid-like nature of the hydrogel matrix allows for T-cell activating antigens (such as CD3/CD28) to be covalently bonded with the hydrogel matrix, or functionalized to form artificial antigen presenting cells, which contact and activate the T-cells as they move through the hydrogel matrix. More broadly adopted, such a fluidic device would enable cells and pathogens to be rapidly produced when such cells are needed for critical therapeutics or countermeasures. The mesofuidic chamber can be incorporated into an affordable polymeric device to provide a disposable, single-use cell culture system for rapid expansion of cells. The device may operate alone or as part of an array of 3D mesofluidic cell expansion devices.

Turning to FIG. 5, another embodiment of a fluidic device 50 is shown for isolating, immobilizing and interrogating pathogens. The fluidic device 50 is capable of accepting a sample at a sample port or inlet 52. The sample may contain a large number of microbes with a subpopulation of potential pathogens. Viable bacteria in the sample are separated using various fluidic methods. Illustrated here, for example, is a coarse separation section 54 that uses asymmetrical flow field-flow fractionation (AF4), and a fine separation section 56 downstream thereof which uses, for example, acoustofluidics. The bacterial cells are delivered in small aliquots to a chamber 58 containing the thermoresponsive microgel particles. Each aliquot is trapped locally onto a small space in the chamber, for example with heating/cooling loops 59, three of which are illustrated here. The bacterial cells are heated to a culturing temperature (e.g., 37° C.) to allow the bacteria or pathogenic cells to grow.

The microgel chamber 58 provides an on-chip host mimicking environment that is useful for interrogating bacterial cells and pathogens. The microgel particles may be decorated with reporter probes ranging from synthetic receptors to live mammalian host cells. Specific cells that generate a signal of interest may be selectively released, for example by selectively cooling a loop 59. This releases the pathogenic bacteria and delivers them to a single cell sequencing operation 60. This ability to rapidly detect unknown pathogens and sequence the expanded bacteria cells allows for the development of effective medical countermeasures before widespread outbreak occurs.

In further contemplated embodiments, thermo-responsive packed microgel particles can be incorporated in a device that combines one or more of the processes of devices 40, 50 in FIG. 4 and FIG. 5. In such embodiments, the device can rapidly detect a new, unknown pathogen, and rapidly generate a specially designed T-cell that can be harvested and administered to the patient to seek out and kill the pathogen in vivo. Therefore, the applications of the present disclosure are widespread and can provide patients with an instant cell mediated immune response to pathogens.

One aspect of the present cell culture systems is that they provide rapid and affordable systems for activating and expanding cells when immediate and quick treatment is critical to the patient's recovery. As illustrated in the examples, the disclosed cell culture systems can provide rapid activation and expansion of T-cells for cell-based immunotherapies used to treat blood cancers, such as leukemia and lymphoma.

Another aspect of the present disclosure is fluidic devices that can be used as a rapid response, medical countermeasure for emerging infectious disease threats. The devices disclosed herein provide the flexibility and agility needed to address emerging and unexpected outbreaks and bioterrorism threats.

Another aspect of the present bioreactors and fluidic devices is a label-free approach for selectively trapping and releasing cells.

### EXAMPLE APPLICATIONS

### Example 1—CAR-T Therapy

Chimeric Antigen Receptor T-Cell (CAR-T) Therapy has the potential to treat numerous difficult-to-drug targets or diseases that subvert traditional therapeutics. CAR-T is already employed in the treatment of blood cancers, such as lymphoma and leukemia. The current treatment requires a time and labor intensive process that involves the steps of separating a patient's T-cells from a blood sample; transfecting the T-cells with a nucleotide to have them express a CAR that will target the CD19 antigen on the surface of a cancerous cell; activating growth of the T-cells using CD3/CD28 antigens or artificial antigen presenting cells (aAPCs); expanding the T-cells in an in vitro culture; and finally administering the T-cells in the original patient as a redirected, cancer-killing cells.

The current CAR-T treatment faces a formidable scaling problem due to the high cost, time and labor requirements. Mass production of similar therapies require significant design and development of new tools. Devices, as described in connection with FIG. 4, can reduce the time, number of steps, and touch points involved in the treatment.

A cell culture system as disclosed herein provides for rapid activation and expansion of T-cells for use in cell-based immunotherapy. The liquid-like nature of the 3D hydrogel matrix—as employed in the device of FIG. 4 or a similar device—allows for T-cell activating antigens (such as CD3/CD28) that are covalently bonded to the hydrogel matrix, or functionalized onto microparticles to form artificial antigen presenting cells, to contact and activate T-cells as the cells move through the matrix. Upon cooling, the liquid-like support contracts and allows the media to become a flowable liquid so expanded T-cells can be harvested. A cell culture system, incorporated as part of a fluidic device, can provide for rapid separation, transfection, and expansion of T-cells used as a platform for the rapid production of medical countermeasures applied against a wide range of discoverable pathogens.

### Example 2—Nerve Replacement

The microgel particles of the present disclosure can be employed as a scaffold for neuronal stem cell models.

Upper limb injuries are among the most frequent causes of disability in military patients, and over fifty percent (50%) of the disabilities are the result of nerve trauma. Peripheral nerve injuries (PNIs) are a significant complication of injuries incurred in the military setting with devastating, long term consequences, which can include sensory deficit (loss of sensory perception, chronic pain, and weakness (reduced strength and motion). PNIs typically accompany radial nerve trauma from a blast injury. While, cell-based therapies are being developed with the potential to stop or reverse nerve damage and the resulting disabilities in patients, manufacturing and scalability issues have prevented translation of these technologies to the patients who need them.

The present disclosure is contemplated for use in nerve replacements designed to stop or reverse nerve damage. For example, by employing the self-healing microgel particle scaffold 14 in the bioreactor 10 of FIG. 3, neural structures can be printed with unprecedented versatility and fidelity. The scaffold enables pre-defined nerve shapes to be seeded in arbitrary geometries (length, diameter, bifurcations, and axonal microstructuring) that are tailored in real-time to particular maladies or neural regeneration needs. The scaffold further enables the pre-defined nerve shapes to be seeded with cell mimicking polymer microgels (CMPMs) that present growth factors, which encourage specific neural growth rates and spatial orientation. A specialized printing ink—comprised of a complex mixture of the microgel particles, growth factors, neuronal stem cells, axons, and oligodendrocytes, for example—can be directly printed onto the growth factor-seeded patterns in a core-sheath or other defined structure that mimics physiological nerve morphology and further encourages growth in a predefined orientation. In one embodiment, the printing ink can include allogenic induced pluripotent stem cells (iPSCs) that are grown in vitro to produce organoid tissue(s). In another embodiment, the printing ink can include neural crest like cells (NCLCs). In a different embodiment, the CMPM can be functionalized with a mucoadhesive and/or attachment promotor. In another embodiment, the printing ink or the macrogel can include a human leukocyte antigen (HLA) to prevent rejection of the organoid tissue implant due to HLA mismatch. The HLA can knock out human iPSCs. Instead, engineered iPSCs can be employed which express CD47 surface proteins to further mitigate potential immunogenic rejection.

Using the bioreactor platforms disclosed herein, nerve structures can be generated with high throughput parallel processing, while leveraging the versatility afforded by 3D printing. In one embodiment, iPSCs may be used to generate nerve replacement structures as a near “off-the-shelf” therapy option for regenerating and/or restoring nerve function in peripheral nerve injuries. The bioreactors enable an organoid tissue (nerve structure), mimicking physiological nerve morphology, to be grown in vitro for implantation into a human patient. The disclosed methods reduce the time required for creating the nerve replacement structures over known tissue regeneration technologies. Parallel arrays of nerve growth niches can be simultaneously processed by first seeding the microgel particles (containing growth factor) into the LLS to form a scaffold, and then dispersing a printing ink into a core-sheath structure about the scaffold.

The structure and electrical functionality of the generated nerves can be assessed in vitro before being implanted into the patient.

There is no contemplated limit to the cell type that can be grown in the disclosed microgel or the organoid tissue that can be replicated by a scaffold formed from the microgel. In one non-limiting example, an embodiment contemplates that schwann cells can be seeded in the microgel for producing myelin proteins for sheath production.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A cell culture system, comprising:
a bioreactor chamber including a liquid-like solid (LLS) growth media;
a scaffold formed from a plurality of microgel particles seeded into the LLS growth media, the microgel particles comprising a thermally responsive hydrogel matrix; and
one or more cells dispersed throughout the scaffold.

2. The cell culture system of claim 1, wherein the hydrogel matrix of the microgel particles has a yield stress of about 10 pascal (Pa).

3. The cell culture system of claim 1, wherein the microgel particles can be displaced with a protrusion force of about 1 piconewton (pN).

4. The cell culture system of claim 1, wherein the cells are allogenic induced pluripotent stem cells (iPSCs).

5. The cell culture system of claim 1, wherein the cells are selected from the group consisting of neuronal stem cells, axons, oligodendrocytes, and combinations thereof.

6. A cell culture system, comprising:
a microfluidic device containing microgel particles in at least one chamber formed in the device, the microgel particles comprising a thermally responsive hydrogel matrix; and
one or more cells mixed with the microgel particles;
wherein the cells expand in the hydrogel matrix to form a cell culture.

7. The cell culture system of claim 6, wherein the device is disposable.

8. The cell culture system of claim 6, wherein the chamber is one in an array of multiplexed chambers formed in the device.

9. The cell culture system of claim 8, wherein a temperature of each chamber is selectively controllable to heat or cool the microgel particles in the chamber.

10. The cell culture system of claim 6, wherein the cell culture system traps or releases the cells in the hydrogel matrix by controlling a temperature of the microgel particles in the chamber.

11. The cell culture system of claim 6, wherein the microfluidic device further includes at least one input channel for delivering the cells to the chamber; or
wherein the microfluidic device further includes at least one output channel for delivering the cell culture from the chamber.

12. A microgel particle comprising a thermally responsive hydrogel matrix.

13. The microgel particle of claim 12, wherein the hydrogel matrix comprises a thermo-responsive polymer that expands at a temperature of about 37° C.

14. The microgel particle of claim 12, wherein the hydrogel matrix behaves as a Bingham plastic.

15. The microgel particle of claim 12, having a particle size on the order of a mammalian cell (10 μm to 100 μm).

16. The microgel particle of claim 12, wherein the hydrogel matrix yields under a stress of about 10 Pascals.

17. The microgel particle of claim 12, further comprising at least one signaling molecule for inducing at least one cellular process selected from the group consisting of: growth; differentiation; activation; taxis; transport; mitosis; apoptosis; and a combination of the above.

18. The microgel particle of claim 12, further comprising a growth factor, protein, exosome, or cell lysate for inducing at least one cellular process selected from the group consisting of: growth; differentiation; activation; taxis; transport; mitosis; apoptosis; and a combination of the above.

19. The microgel particle of claim 12, further comprising a growth factor that encourages a specific cellular growth rate and spatial orientation.

20. The microgel particle of claim 12, further comprising an antigen that activates a mammalian cell moving through the hydrogel matrix.
